(19) United States
(12) Patent Application Publication
(10) Pub. No.: US 2022/0327484 A1
(43) Pub. Date: Oct. 13, 2022

(54) SYSTEM AND METHOD FOR CLINICAL PRACTICE AND HEALTH RISK REDUCTION MONITORING

(71) Applicant: Brian Gale, Bronx, NY (US)

(72) Inventor: Brian Gale, Bronx, NY (US)

(21) Appl. No.: 17/838,731

(22) Filed: Jun. 13, 2022

Related U.S. Application Data
(62) Division of application No. 12/905,980, filed on Oct. 15, 2010.
Application No. 12/905,980 is a continuation-in-part of application No. 12/408,686, filed on Mar. 21, 2009.
Application No. 12/408,686 is a continuation-in-part of application No. 12/361,081, filed on Jan. 28, 2009, now abandoned.
(60) Provisional application No. 61/038,729, filed on Mar. 21, 2008.
(60) Provisional application No. 61/252,100, filed on Oct. 15, 2009.
(60) Provisional application No. 61/252,097, filed on Oct. 15, 2009.
(60) Provisional application No. 61/255,773, filed on Oct. 28, 2009.
(60) Provisional application No. 61/262,431, filed on Nov. 18, 2009.
(60) Provisional application No. 61/297,773, filed on Jan. 24, 2010.
(60) Provisional application No. 61/299,268, filed on Jan. 28, 2010.

(51) Int. Cl. G06Q 10/10; G16H 40/20; G16H 10/40; G16H 10/60

(52) U.S. Cl. CPC G06Q 10/10; G16H 40/20; G16H 10/40; G16H 10/60; G16H 40/40

(57) ABSTRACT

A System and Method for monitoring risk reduction activities in one or more medical practice settings and environments is described. The system automatically monitors communications and electronic medical records to determine a risk metric indicating compliance with responding to a critical test result by acknowledging it or otherwise acting upon the message.

### PRIORITY CLAIM

This application is a Divisional of, claims priority to and incorporates by reference U.S. patent application Ser. No. 12/905,980 filed on Oct. 15, 20101, which itself claims priority to and herein incorporates by reference in their entirely U.S. Provisional Patent Application No. 61/252,100 filed on Oct. 15, 2009; U.S. Provisional Patent Application No. 61/252,097 filed on Oct. 15, 2009; U.S. Provisional Patent Application No. 61/255,773 filed on Oct. 28, 2009; U.S. Provisional Patent Application No. 61/262,431 filed on Nov. 18, 2009; U.S. Provisional Patent Application No. 61/297,773 filed on Jan. 24, 2010; U.S. Provisional Patent Application No. 61/299,268 filed on Jan. 28, 2010; and is a Continuation in Part to U.S. patent application Ser. No. 12/408,686, filed on Mar. 21, 2009 which further claims priority to both provisional application 61/038,729, filed on Mar. 21, 2008 and as a continuation in part to U.S. patent application Ser. No. 12/361,081, filed on Jan. 28, 2009.

### SUMMARY OF THE INVENTION

The Joint Commission hospital accreditation organization has made Critical Test Reporting a priority in its National Patient Safety Goals. Court rulings now indicate that reporting physicians (who perform diagnostic procedures and provide consultations) may have a duty to communicate critical findings to referring clinicians as well as patients and from clinicians themselves to patients. The advent of electronic medical records (EMR) enables physicians and health care facilities to document health care activity with increasing precision and reliability. In some cases, the data being entered into the electronic medical records or the operation of diagnostic exam request and test result creation and delivery is compromised by a lack of prompt attention to incoming or outgoing report messages or failure to act on those reports. In another embodiment, the invention applies data integrity rules to check that as participating users of the system send or receive test result message, or enter data into medical records, the timing of such activities meet a predetermined threshold of promptness associated with the diagnosis. This way the integrity of the message data and timing of communication is not compromised and may be relied upon for monitoring risk reduction activities or activities that are elevating risk. Equally important is monitoring whether critical test result notifications have been acted upon, which may entail a variety of actions. At the same time, the medical field has been transitioning to use of digital data systems for inputting and maintaining patient medical records and conducting such critical test result reporting.

One particular problem arises from the use of text data input into a patient record by a physician or other care-giver that is relatively unstructured text data. The invention relates to processing such unstructured text data in order to determine whether the data embodies or describes a critical test result report and further whether in the patient record data there is an indication that the report was acted upon. A clinician acting upon a critical test result report may include acknowledging they received or reviewed the message, or that they issued orders based on the incoming test result. Tracking that clinicians are complying with the need to act upon these messages reduces the risk of malpractice. Fort his reason, there is a need to process unstructured EHR data in order to determine whether a critical test result was received and if so, whether the clinician acted upon it in some way. This may be accomplished by key word searching, key word frequencies, natural language parsing and other techniques. The invention relates to a method executed by a computer system for extracting from a critical communication document data a data value representing a logic state that it was acted upon, said method comprising: retrieving into the computer system at least one document data comprised of an at least one corresponding text data comprising an at least one medical data; using a first word statistical frequency analysis process to automatically extract a corresponding at least one first set of at least one keywords from the at least one text data; using the extracted at least one first set of at least one keywords to automatically determine whether the at least one document data is comprised of a corresponding at least one critical communication document; using a second word statistical frequency analysis process to automatically extract from the at least one determined critical communication document data a corresponding at least one data value representing the logic state that the at least one critical communication was acted upon; and storing the extracted data value in computer memory.

### BRIEF DESCRIPTION OF THE DRAWINGS

FIG. 1: Flow chart presenting steps for a critical test result monitoring systems that tracks critical communications residing in medical records and referring physician acting upon them.

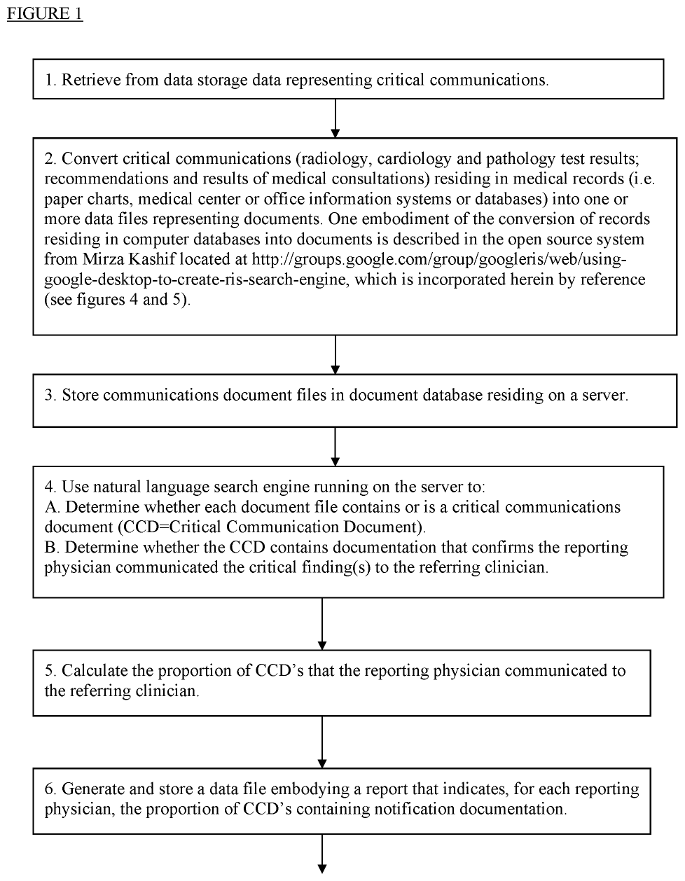

FIG. 1A: Continued flowchart from flowchart in FIG. 1.

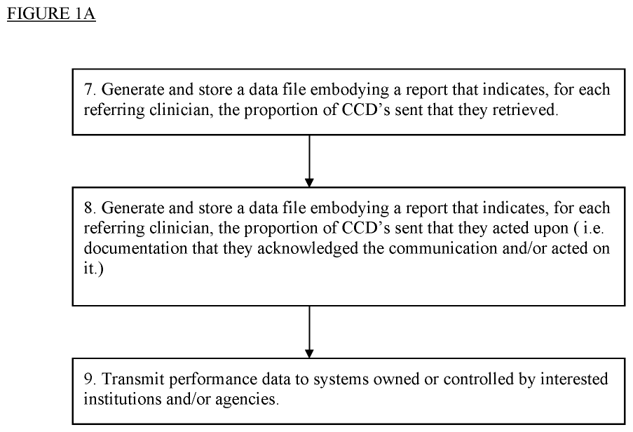

FIG. 2: Flow chart presenting steps for a monitoring system that tracks outpatient status and records and transmits ADT performance data to interested institutions and/or agencies.

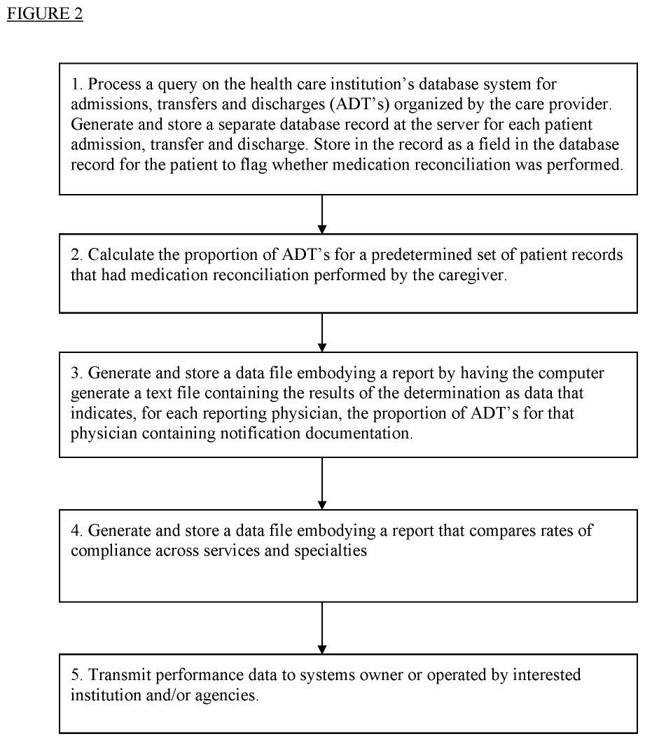

FIG. 3: Flow chart presenting steps for a database warehouse system that allows users to access documentation of risk reduction activity in electronic medical records and other electronic databases.

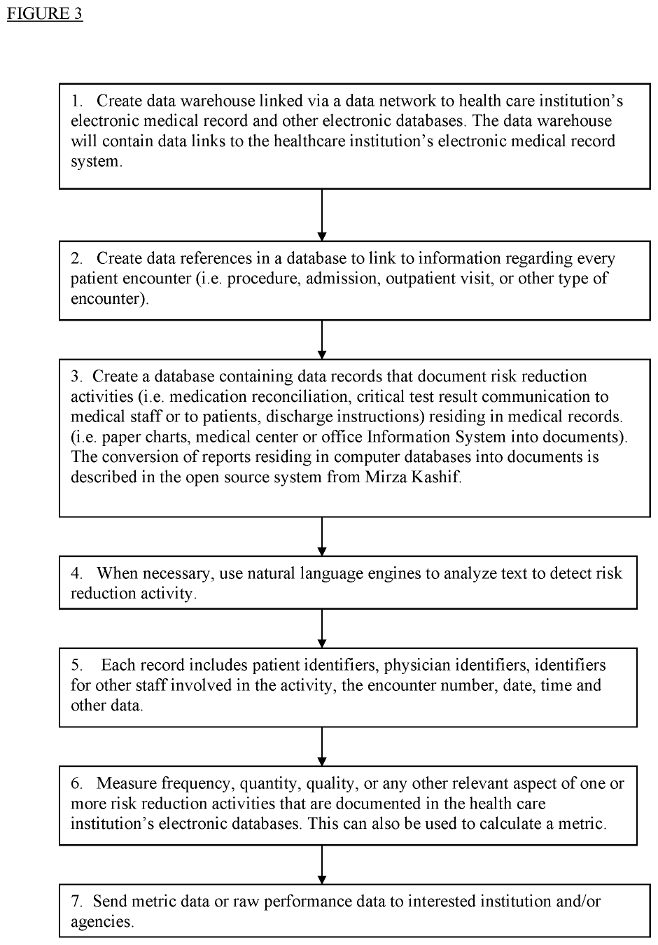

### DESCRIPTION OF THE PREFERRED EMBODIMENTS

Critical test reporting systems and risk mitigation systems are disclosed in U.S. patent applications Ser. No. 12/408,686, filed on Mar. 21, 2009, 61/252,100 filed on Oct. 15, 2009, 61/252,097 filed on Oct. 15, 2009, 61/262,431 filed on Nov. 18, 2009 and 61/297,773 filed on Jan. 24, 2010 all of which are incorporated herein by reference.

In one embodiment, the system starts by:
1. converting critical communications (radiology, cardiology and pathology test results;

recommendations and results of medical consultations) residing in medical records (i.e. paper charts, laboratory medical center or office information systems, EMR or diagnostic test result databases) into one or more data files representing documents or data records in a database.
2. storing communications document files in document database residing on a server.
3. using text processing techniques running on the computer to:
   A. Determine whether each document file contains or is a critical communications document (CCD=Critical Communication Document) In this application “Critical Communication” would include any result that is reportable or should be reported, whether or not the level of urgency is considered “critical” at a particular time.
   B. Determine whether the CCD contains documentation that confirms the reporting physician communicated the critical finding(s) to the referring clinician.
4. Calculate the proportion of CCD's that the reporting physician communicated to the referring clinician.
5. Generate and store a data file embodying a report that indicates, for each reporting physician, the proportion of CCD's containing notification documentation.

In another embodiment the additional step of:
6. automatically generating from text processing of the data a list of referring clinicians, or from the referring clinician field for all of the diagnostic test reports. This can be generated from the database records of critical result notifications. In one embodiment, a data field in the record lists the name of the referring health care provider. The list of notified referring providers may be used to calculate a metric regarding their compliance to malpractice insurers, certification agencies, other interested entities, or by the health care institution itself.
7. In one embodiment, the result message to the referring physician may be matched against some action taken by the referring physician, or simply whether the referring physician retrieved the message.
8. From this data, the system can calculate and store a data file embodying a report that indicates, for each referring clinician, the proportion of CCD's sent to them that they retrieved.

Furthermore, the system can then analyze the text data and data base records to
1. calculate and store a data representing the condition that the text is a CCD and
2. calculate and store a data representing the condition that the CCD was was acted upon in some way or not.

The file may indicate, for each referring clinician, the proportion of CCD's sent that they acted upon (i.e. documentation that they acknowledged the communication and/or acted on it by issuing further orders).

The system may also transmit performance data to systems owned or controlled by interested institutions and/or agencies. Transmission may be accomplished by an automatically generated email, FTP (File Transfer Protocol) or any other means of moving digital data from one system to another. The transmission may be the result of a request by such interested institution or agency that is submitted to the computer system. In another embodiment, the system automatically transmits the data at predetermined times.

Where information resides in text data comprising email traffic, the software embodying the invention, will, when running on a computer, request data files that represent the email messages. The software will parse the email data to find who the sender, receiver, subject and contents of the message. In one embodiment, keyword searching can be used to determine what type of message the email was. For example, certain keywords used by the physician or practice may be correlated with a critical test result report or finding. This keyword set can be used by the system to automatically determine if the text data is a CCD. If the system determines that it is a CCD, the system can then use a set of keywords to analyze later actions in the patient data record that indicate that the report was acted upon in some way.

The software will generate a database record that indicates when the message was sent, when it was received, the sender, the recipient and the subject matter. In another embodiment, the software interfaces with a database that holds certain patient diagnostic data. The software will, when running, format requests and submit them to the database in accordance with typical database languages, for example, SQL. The database will return results that the software then uses to tabulate the information in the form it uses it. In this embodiment, the critical communications may have a relevant flag in a data field, for example identifying the message as a test result. In yet another embodiment, the software can parse data files that are contain text in repetitive patterns or fields, in order to populate a database with the relevant information.

The system will comply with the requirements of the Health Insurance Portability and Accountability Act (HIPAA) and the Health Insurance Portability and Accountability Act (HIPPA) and the Healthcare Information Technology for Economic and Clinical Health Act (HITECH). One means of compliance is for the data that is provided to be scrubbed of any actual patient identity data in order that it be anonymous. In that embodiment, the patient data fields that are specified for patient name, address or social security number are deleted. In another embodiment, a patient serial number can be assigned that is a random number in order that a specific patient record be used individually, but without any known mapping from the patient name to the random number, sometimes referred to as “aliasing”.

2. Create data references in a database to link to information regarding every patient encounter (i.e. procedure, admission, outpatient visit, or other type of encounter).

3. Create a database containing data records that document risk reduction activities (i.e. medication reconciliation, critical test result communication to medical staff or to patients, discharge instructions) residing in medical records. (i.e. paper charts, medical center or office Information System documents, emails, or other data messages). The conversion of reports residing in computer databases into documents is described in the open source system from the Mirza Kashif reference incorporated by reference above.

One problem is that often the patient data record is comprised of a clinician's report that is unstructured text that essentially is the entire report in prose. The system can use use natural language engines or other heuristics or algorithms to analyze text to detect the state that the report is a CCD. This may be accomplished by key word searching, key word frequencies, natural language parsing and other techniques. In one embodiment, the program holds a list of important groups of key words, where each group is relevant to a known subject and a frequency key where for a given group, a frequency of use of that member word is expected. The program then searches for all the key words in the text and tallies the frequency of use of the word in the text. Finally, the program performs a best-fit analysis to determine which group of keywords use frequency matches the closest or sufficiently matches as compared to a predetermined quality value for fit. This can be performed by linear regression or R square analysis or similar known methods of determining the quality of fit or correlation between two statistical results. The program then updates its database to indicate the text is relevant to the subject matter associated with that group. Additional heuristics may be applied to a group whereby two words in the group are frequently used in the same sentence. The measure would then be the number of times the one word appears in the same sentence as the other. This statistic can be used to improve the distinctiveness of word groupings. Where more than one group may appear relevant, the program can prompt a human user to specify the outcome. Another method would be to generate a list of critical findings from those test reports that resulted in notifications to the referring clinician, or were flagged as a critical test result, either via language within the report, or via database flag set by the diagnostic physician, staff, or equipment or a data flag set or data field entered in the message in accordance with a protocol. This data may be used as a truth set for determining the best keywords to use to detect the presence of a CCD.

Each record in the database includes patient identifiers, physician identifiers, identifiers for other staff involved in the activity, the encounter number, date, time and other data.

Measure frequency, quantity, quality, or any other relevant aspect of one or more risk reduction activities that are documented in the health care institution's electronic databases. This can also be used to calculate a metric. The data can be stored in the database created.

For each healthcare provider or patient, obtain data from the database and use it to calculate a metric data value. The metric data value can represent one or more conditions of compliance with a base-line requirement for response to critical test result report messages.

8. Send metric data or raw performance data to interested institution and/or agencies. Alternatively, the data may be used by the organization for management of systems and personnel. In this embodiment, the metric data or raw performance data is used by a system internally to report compliance statistics to clinical care managers.

In another embodiment, additional metrics can be determined, calculated and used, for example:

Incidence of check list use: For every central line placed, how often did the staff follow the procedure checklist to reduce the incidence of infections.

How often did the facility provide discharge instructions to the patients?

For critical results, how often did the staff at the facility communicate the results directly to the patient or to the referring physician, acknowledge receipt or issue orders in response to the report.

These types of metrics can be numerically calculated in a variety of ways. In one embodiment, the number of procedure checklists that are cited can be divided by the number of procedures of the same type to determine a percentage. Similarly, the denominator can be the total number of procedures of all types. The denominator can be determined based on a pre-determined amount of time for example, procedures during a particular week, month, quarter or year. In another embodiment, the metric can indicate frequency, for example the average rate of discharge instructions being cited in the EMR data as compared to the rate of discharges during the same time period. In another embodiment, the system can calculate the frequency of facility communications of test results as compared to the frequency of tests conducted. The frequencies can be determined on a time period basis. In addition, the frequencies can be determined by examining the same class of test or the same category of intended message recipient. Healthcare providers may be benchmarked against similar specialists practicing in similar settings.

Given the importance of compliance and its impact on patient safety, there is a need to check whether the data in the system is dependable from a risk assessment standpoint. Consider the following example, on Jan. 13, 2010, a diagnostic physician interprets a diagnostic imaging exam and notices an abnormal test finding. He fails to appropriately communicate the finding to the patient's physician. On May 1, 2010, the diagnostic physician learns that, as a result of his failure to communicate the result, the patient's physician failed to diagnosis and treat a serious condition, and that the condition has worsened. Fearing a malpractice liability lawsuit, the diagnostic clinician might be tempted to create false documentation that he did communicate the abnormal test result at the time of exam on Jan. 13, 2010. If the data record of the Jan. 13, 2010 communication is sent to the CTRM monitor database on May 1, the system could flag it as suspicious, or reject the data. In this case the system checks the cited date in the data record with the date of the actual change in the database.

The System will use the following techniques to assess the reliability of the data received:

Periodic or Data Transfers—The system will execute periodic data transfers, in one embodiment, a daily data transfer, by and among the facility, practitioner, 3rd party service provider, or other source of practice data. This precludes retrospective manipulation of data. That is because the data being used to monitor physician activity becomes off-limits by the end of the day. In another embodiment, the data transferred hourly.

System operators could adjust the system's time tolerance interval (i.e. 1 minute versus 1 day, or 1 month). This would enable them to calibrate the system's rejection parameters to the data transfer interval.

Test Data against Business Rules—The practice data transferred into the CTRM Monitor system reflects normal practice patterns. For example, a notification of an abnormal diagnostic test result is sent at a given time. The time stamp is time data that the system inserts into the data structure or data record constituting the message, without physician adjustment, rather than data input by the physician. Sometime later, a clinician retrieves the abnormal test result message from the system. This can result in an additional time stamp inserted into the data record. The event time stamps in the data will be checked against certain logic based on expected sequencing and time tolerance intervals. If the message retrieval time is earlier than the notification time, the system could flag the data record as suspicious, or reject it.

Normative Data—The system could trend practice activity by type of practitioner and practice setting. For example, the system could determine the median number of abnormal test result messages generated by neuroradiologists in urban academic hospitals. The system could use statistical tests (i.e 95% confidence intervals) to determine if the practice pattern of a given practitioner is significantly different from those of most similar practitioners.

### Data Testing

Insurers and other interested parties rely on data from this system that demonstrates clinical activity that precludes or reduces risk of certain types of medical misadventures. This module is designed to confirm the reliability of the clinical data provided. The system is designed to detect false documentation. This could arise from deliberate data manipulation, technical error, data corruption, or other causes. Any interested stake holder may be interested in manipulating the clinical or CTRM data. These include (but are not limited to): the diagnostic physician (or lab), the receiving clinician, and the healthcare facility. In the case of abnormal test result notification, the community standard requires the diagnostic physician to communicate directly to the referring clinician. The normal sequence of events are:

(1) Diagnostic physician interprets exam and identifies abnormal finding

(2) Diagnostic physician communicates the abnormal test results to the referring clinician

(3) Referring clinician orders additional medical procedures in response.

The data elements typically required to document appropriate notification are:

1—Date/Time of notification of abnormal or emergent test result

2 13 Content of notification

3—Identify the clinician that received the notification

4—Date/Time clinician obtained the notification

Alternatively, the diagnostic physician can use a Critical Test Results Management (CTRM) system to deliver the notification. When using a CTRM system, the diagnostic physician creates a message containing the abnormal test result that is recorded in the system, one embodiment of the system records time stamps when:

(a) Message Creation: The Diagnostic physician creates the abnormal test result notification

(b) Notification: The system sends notification that there is an abnormal test result to the referring clinician

(c) Repeat Notification: If the referring clinician fails to retrieve the message after the first notification, the CTRM system sends a repeat notification

(d) Notification Escalation: If the referring clinician fails to retrieve the abnormal test result message within a specified compliance interval (the length of the compliance interval depends on the urgency category of the message), the system escalates the message to a backup physician.

(e) Message Retrieval: the referring clinician accesses the CTRM system to retrieve the abnormal test result message.

(f) Message Retrieval Completion: the referring clinician listens to the message in its entirety.

(g) Read Back: The referring clinician repeats the message in order to document that she understands the finding.

(h) Return message: the referring clinician may elect to send a message back to the diagnostic clinician.

Each of these events may be documented as data in the diagnostic procedure report, or in a database that documents notifications of abnormal test results. In one embodiment, the database could reside in the electronic medical record or at the CTRM service vendor. In another embodiment, the event time stamps are data records in a relational database that are linked to the EMR or linked to the diagnostic reports themselves. These data records may be stored in a separate server housed under the control of the monitoring system provider. In another embodiment, the data may be obtained by query of an electronic medical record (EMR). For example, the system may analyze radiology reports in the EMR to determine if the report has been finalized and to detect documentation of abnormal test result notification. Once the report is finalized, a data record is created, identified with a unique serial number. The sequence of events of the abnormal test result notification (or lack thereof) are recorded in the database record for that report.

Certifying/ratings agencies and liability insurance carriers need reliable data in order to appraise the practitioner and/or healthcare facility. The DATA TESTER module of the system evaluates the reliability of database records that document critical test result notification and message retrievals. The system is designed to access or import documentation of abnormal test result notification and other relevant data into a database. The system imports data from multiple sources: for example, CTRM services, electronic health records (EMR), as well as paper medical records that can either be scanned with optical character recognition systems known in the art or the data hand entered. The latter requires manual data entry.

Each abnormal test result can be identified with a unique number. That ID # may be associated with one or several database records of notifications containing time stamps.

In one illustrative embodiment:

| Abnormal | | | | | |
|---|---|---|---|---|---|
| Test Result # | Notification Time/Date | Sender | Recipient | Retrieval Time/Date | Retrieval Completion |
| 00001 | Jan. 21, 2010 09:35 | Nelson | Zamboni | — | |
| 00001 | Jan. 21, 2010 09:40 | Nelson | Zamboni | — | |
| 00001 | Jan. 21, 2010 09:45 | Nelson | Zamboni | — | |
| 00001 | Jan. 21, 2010 09:50 | Nelson | Gibbons | Jan. 21, 2010 09:52 | Jan. 21, 2010 12:10 |
| 00001 | Jan. 21, 2010 09:55 | Nelson | Zamboni | — | |

In this case, Dr. Nelson used a CTRM system to send the original notification of Abnormal Test Result #1 on 1/21/10 @ 09:35. The system sent the first notification at 9:35. With no response, the CTRM system sent additional notifications every 5 minutes, and then escalated the notification to Dr. Gibbons. Dr. Gibbons answered the message immediately. After that, the system stopped sending notifications. Dr Gibbons started to listen at 9:52 but didn't complete listening to the entire message until 12:10.

There are several scenarios in which the database record documenting abnormal test result notification could be false. Some of these data relationships go beyond CTRM. These scenarios involve the relationship between events documented in the medical record (i.e. in progress notes). These may documented in the electronic medical record (EMR), using DICOM, HL7 W3C and other medical data interchange standard formats, subject to security restrictions. The Data Tester applies one or more data integrity rules against the notification record(s). If the time stamps and other data are inconsistent with the rules, the notification record is flagged as suspicious. Several exemplary rules are presented below.

Delayed Test Order (Request): Clinician examines patient, but fails to request the diagnostic exam in a timely manner, i.e. within a maximum period of time. Data Tester compares clinical visit date/time to diagnostic procedure order and performance time. If the time intervals are longer than a pre-defined compliance interval, the record is flagged as suspicious. Application of the rule checks whether an diagnostic examination is ordered by the clinical physician after the diagnosis was already known and entered into the record: A diagnosis is known to the referring clinician. She orders a diagnostic test in an attempt to create a false documentation that the diagnosis became apparent after the new diagnostic procedure. The Data Tester compares the Electronic Medical Record documentation of the abnormal diagnosis to the date/time the diagnostic exam was ordered. If the time stamp associated with the exam order comes after the diagnosis itself was entered into the EMR, the record is flagged as suspicious.

Delayed Test Interpretation: In this case, a diagnostic test is performed in a timely manner, but there is a delay in interpretation. The system compares the time stamps associated with the procedure date/time and the report date/time to the notification date/time. Depending on the severity of the diagnosis, if the notification interval surpasses a compliance interval, the record is flagged as suspicious. The system will classify families of diagnoses with a ranges of pre-determined interval thresholds. When the test is conducted, the type of diagnostic test is extracted from the data record and then mapped to the appropriate time interval threshold to apply as the test.

Delayed Notification of Abnormal Result: In this case, the Diagnostic clinician interprets the diagnostic procedure, but fails to communicate the abnormal test result. Months later, she learns that the patient was harmed because of a failure or delay in diagnosis. At that time, she sends notification to the referring clinician. The Data Tester compares the time stamps associated with the procedure date/time and report date/time to the notification date/time. Depending on the severity of the diagnosis, if the notification interval surpasses a compliance interval, the record is flagged as suspicious. When the test is conducted, the type of diagnostic test is extracted from the data record and then mapped to the appropriate time interval threshold to apply as the test.

Fabricated Documentation: In this case, the Diagnostic clinician fails to communicate abnormal test result. Diagnostic physician enters back dated, false documentation of abnormal test result notification into the database. The Data Tester compares abnormal result serial number and the date record was created in database to the report date. If the notification record was created after the report date, or if the notification record ID# is out of sequence, the record is flagged as suspicious. In one embodiment, the system automatically tags the creation date of each of the report data record and the notification record. This data is not alterable by the users of the system. In another embodiment, the numerical identifiers associated with a patient are issued in a pre-determined sequence. In one embodiment, a first predetermined set of digits of in the number identify the patient and a second predetermined set of digits identify the order of action in the patient diagnostic process. These numbers are not changeable by the system users, rather, they are typically generated automatically by the system itself.

Delayed retrieval of test result message: In this scenario, the Data Tester determines how long it took for the clinical physician to retrieve the notification, or the retrieval interval. If interval is prolonged, record is flagged as suspicious. In this case, the Diagnostic Physician sends notification, but the Clinical Physician fails to retrieve message within a pre-determined time interval threshold. As a result the Electronic Medical Record is flagged as suspicious.

In another case, the DP Sends notification, CP fails to retrieve message, but the data record later changes to indicate message was retrieved on time. Since the data record was previously retrieved any “after the fact” change would suggest documentation tampering. Record would be flagged as suspicious.

Corruption of EMR data: This can result in several suspicious data elements. One logic rule tests: for example, is the physician's name known to the system as being associated with the system, or the patient or the clinician or the diagnostician?

Sequence Rule Compliance: For this case a logic rule tests: Do the time stamps for the sequential events of an abnormal test result notification appear in the correct sequence? The system will confirm that the notification time stamp occurs before the message retrieval time stamp.

By “send” it is meant that a data message is formulated and transmitted by digital data networks to a computer operated by or on behalf of a clinician or diagnostician or other party. For example, when an abnormal test result is entered into the system, the system can send an email to a designated email address associated with the clinical physician. The logic rules are applied by first querying the relevant data in the patient data record or retrieving it by parsing the data message. The computer program executing the logic rule then compares that one or more data values to one or more other retrieved data values to return a logical or numerical result. This value may then used by the program to cause, in appropriate cases, a change in program logic that results in the system causing a data message being transmitted. Statistical analysis is performed by applying a database query to obtain one or more relevant data values from data records that meet the query requirement. These data values can be organized as a table that is stored as a data structure in a computer. The data structure may by parsed and the data values input into calculations that produce mean, average, standard deviation and similar statistical measures for the sample values. Other fields in the data record may include: date, time and type of procedure, patient age, patient weight, patient body habitus. These doses can be numerically compared by the equipment to typical doses given the procedure type and patient characteristics by comparing the stored dosage data with a predetermined normative threshold value stored elsewhere in the system. The data can also be used as a basis for statistical sampling of use of radiological treatment, with a variety of basis in order to come up with baseline threshold values. In one embodiment, the system will use typical database query methodologies to tabulate all of the dosages for a particular body part. This may be further filtered by patient sex, weight or other characteristics. Then, the system can calculate an average, a mean or other value that can be used as the predetermined comparison threshold. In another embodiment, the predetermined threshold is input into the system and stored as a value.

Operating Environment

The system is typically comprised of a central server or servers that are connected by a data network to a user's computer or device. The central server may be comprised of one or more computers connected to one or more mass storage devices. The precise architecture of the central server does not limit the claimed invention. Further, the user's computer may be a laptop or desktop type of personal computer. It can also be a cell phone, smart phone or other handheld device, including a tablet. The precise form factor of the user's computer does not limit the claimed invention. Examples of well-known computing systems, environments, and/or configurations that may be suitable for use with the invention include, but are not limited to, personal computers, server computers, hand-held computers, laptop or mobile computer or communications devices such as cell phones, smart phones, and PDA's, multiprocessor systems, microprocessor-based systems, set top boxes, programmable consumer electronics, network PCs, minicomputers, mainframe computers, distributed computing environments that include any of the above systems or devices, and the like. Indeed, the terms “computer,” “server,” and the like may be used interchangeably herein, and may refer to any of the above devices and systems.

The user environment may be housed in the central server or operatively connected to it remotely using a network. In one embodiment, the user's computer is omitted, and instead an equivalent computing functionality is provided that works on a server. In this case, a user would log into the server from another computer over a network and access the system through a user environment, and thereby access the functionality that would in other embodiments, operate on the user's computer. Further, the user may receive from and transmit data to the central server by means of the Internet, whereby the user accesses an account using an Internet web-browser and browser displays an interactive web page operatively connected to the central server. The server transmits and receives data in response to data and commands transmitted from the browser in response to the customer's actuation of the browser user interface. Some steps of the invention may be performed on the user's computer and interim results transmitted to a server. These interim results may be processed at the server and final results passed back to the user computer.

The Internet is a computer network that permits users operating a computer device to interact with computer servers located remotely and to view content that is delivered over the network from the remote servers to the computer device as data files or data streams. In one kind of protocol, the servers present webpages that are rendered on the user's device using a local program known as a browser. The browser receives one or more data files from the server that are displayed on the user's computer screen. The browser seeks those data files from a specific address, which is represented by an alphanumeric string called a Universal Resource Locator (URL). However, the webpage may contain components that are downloaded from a variety of URL's or IP addresses. In addition, the data received from a URL may be a data stream rather than a single file. In one embodiment, the browser interacts with a server using the URL using the HTTP or HTTPS protocol. In addition, the browser may send data to the server denoted by the URL by appending data that gets sent to the server into the HTTP or HTTPS request that is comprised of the URL. The server receiving the HTTP or HTTPS request may use the data payload contained in the request in order to process the request. A website is a collection of related URL's, typically all sharing the same root address or under the control of some entity. In one embodiment different regions of the simulated space displayed by the browser have different URL's. That is, the webpage encoding the simulated space can be a unitary data structure, but different URL's reference different locations in the data structure. The user computer can operate a program that receives from a remote server a data file that is passed to a program that interprets the data in the data file and commands the display device to present particular text, images, video, audio and other objects. In some embodiments, the remote server delivers a data file that is comprised of computer code that the browser program interprets, for example, scripts. The program can detect the relative location of the cursor when the mouse button is actuated, and interpret a command to be executed based on location on the indicated relative location on the display when the button was pressed. The data file may be an HTML document, the program a web-browser program and the command a hyper-link that causes the browser to request a new HTML document from another remote data network address location. The HTML can also have references that result in other code modules being called up and executed, for example, Flash or other native code. Alternatively, the data file returned may conform to a hyper text format like XML.

The invention may also be entirely executed on one or more servers. A server may be a computer comprised of a central processing unit with a mass storage device and a network connection. In addition a server can include multiple of such computers connected together with a data network or other data transfer connection, or, multiple computers on a network with network accessed storage, in a manner that provides such functionality as a group. Practitioners of ordinary skill will recognize that functions that are accomplished on one server may be partitioned and accomplished on multiple servers that are operatively connected by a computer network by means of appropriate inter process communication. In one embodiment, a user's computer can run an application that causes the user's computer to transmit a stream of one or more data packets across a data network to a second computer, referred to here as a server. The server, in turn, may be connected to one or more mass data storage devices where the database is stored. In addition, the access of the website can be by means of an Internet browser accessing a secure or public page or by means of a client program running on a local computer that is connected over a computer network to the server. A data message and data upload or download can be delivered over the Internet using typical protocols, including TCP/IP, HTTP, TCP, UDP, SMTP, RPC, FTP or other kinds of data communication protocols that permit processes running on two respective remote computers to exchange information by means of digital network communication. As a result a data message can be one or more data packets transmitted from or received by a computer containing a destination network address, a destination process or application identifier, and data values that can be parsed at the destination computer located at the destination network address by the destination application in order that the relevant data values are extracted and used by the destination application. The precise architecture of the central server does not limit the claimed invention. In addition, the data network may operate with several levels, such that the user's computer is connected through a fire wall to one server, which routes communications to another server that executes the disclosed methods.

The server can execute a program that receives the transmitted packet and interpret the transmitted data packets in order to extract database query information or to process a request using data comprising the data packets. The server can then execute the remaining steps of the invention by means of accessing the mass storage devices to derive the desired result of the query or requested process. Alternatively, the server can transmit the query information or request processing to another computer that is connected to the mass storage devices, and that computer can execute the invention to derive the desired result. The result can then be transmitted back to the user's computer by means of another stream of one or more data packets appropriately addressed to the user's computer. In addition, the user's computer may obtain data from the server that is considered a website, that is, a collection of data files that when retrieved by the user's computer and rendered by a program running on the user's computer, displays on the display screen of the user's computer text, images, video and in some cases outputs audio. The access of the website can be by means of a client program running on a local computer that is connected over a computer network accessing a secure or public page on the server using an Internet browser or by means of running a dedicated application that interacts with the server, sometimes referred to as an “app.” The data messages may comprise a data file that may be an HTML document (or other hypertext formatted document file, like XML), commands sent between the remote computer and the server and a web-browser program or app running on the remote computer that interacts with the data received from the server. The command can be a hyper-link that causes the browser to request a new HTML document from another remote data network address location. The HTML can also have references that result in other code modules being called up and executed, for example, Flash, scripts or other code. The HTML file may also have code embedded in the file that is executed by the client program as an interpreter, in one embodiment, Javascript. As a result a data message can be a data packet transmitted from or received by a computer containing a destination network address, a destination process or application identifier, and data values or program code that can be parsed at the destination computer located at the destination network address by the destination application in order that the relevant data values or program code are extracted and used by the destination application.

The invention may also be practiced in distributed computing environments where tasks are performed by remote processing devices that are linked through a communications network. In a distributed computing environment, program modules may be located in both local and remote computer storage media including memory storage devices. Practitioners of ordinary skill will recognize that the invention may be executed on one or more computer processors that are linked using a data network, including, for example, the Internet. In another embodiment, different steps of the process can be executed by one or more computers and storage devices geographically separated by connected by a data network in a manner so that they operate together to execute the process steps. In one embodiment, a user's computer can run an application that causes the user's computer to transmit a stream of one or more data packets across a data network to a second computer, referred to here as a server. The server, in turn, may be connected to one or more mass data storage devices where the database is stored. The server can execute a program that receives the transmitted packet and interpret the transmitted data packets in order to extract database query information. The server can then execute the remaining steps of the invention by means of accessing the mass storage devices to derive the desired result of the query. Alternatively, the server can transmit the query information to another computer that is connected to the mass storage devices, and that computer can execute the invention to derive the desired result. The result can then be transmitted back to the user's computer by means of another stream of one or more data packets appropriately addressed to the user's computer. In one embodiment, a relational database may be housed in one or more operatively connected servers operatively connected to computer memory, for example, disk drives. In yet another embodiment, the initialization of the relational database may be prepared on the set of servers and the interaction with the user's computer occur at a different place in the overall process.

The method described herein can be executed on a computer system, generally comprised of a central processing unit (CPU) that is operatively connected to a memory device, data input and output circuitry (TO) and computer data network communication circuitry. Computer code executed by the CPU can take data received by the data communication circuitry and store it in the memory device. In addition, the CPU can take data from the I/O circuitry and store it in the memory device. Further, the CPU can take data from a memory device and output it through the IO circuitry or the data communication circuitry. The data stored in memory may be further recalled from the memory device, further processed or modified by the CPU in the manner described herein and restored in the same memory device or a different memory device operatively connected to the CPU including by means of the data network circuitry. In some embodiments, data stored in memory may be stored in the memory device, or an external mass data storage device like a disk drive. In yet other embodiments, the CPU may be running an operating system where storing a data set in memory is performed virtually, such that the data resides partially in a memory device and partially on the mass storage device. The CPU may perform logic comparisons of one or more of the data items stored in memory or in the cache memory of the CPU, or perform arithmetic operations on the data in order to make selections or determinations using such logical tests or arithmetic operations. The process flow may be altered as a result of such logical tests or arithmetic operations so as to select or determine the next step of a process. For example, the CPU may obtain two data values from memory and the logic in the CPU determine whether they are the same or not. Based on such Boolean logic result, the CPU then selects a first or a second location in memory as the location of the next step in the program execution. This type of program control flow may be used to program the CPU to determine data, or select a data from a set of data. The memory device can be any kind of data storage circuit or magnetic storage or optical device, including a hard disk, optical disk or solid state memory. The JO devices can include a display screen, loudspeakers, microphone and a movable mouse that indicate to the computer the relative location of a cursor position on the display and one or more buttons that can be actuated to indicate a command. The JO device may also be sensors that detect haptic motion, fingers scrolling or touching a screen, fingerprint scanners, cameras or other interactivity mechanisms.

The computer can display on the display screen operatively connected to the I/O circuitry the appearance of a user interface. Various shapes, text and other graphical forms are displayed on the screen as a result of the computer generating data that causes the pixels comprising the display screen to take on various colors and shades or brightness. The user interface may also display a graphical object referred to in the art as a cursor. The object's location on the display indicates to the user a selection of another object on the screen. The cursor may be moved by the user by means of another device connected by I/O circuitry to the computer. This device detects certain physical motions of the user, for example, the position of the hand on a flat surface or the position of a finger on a flat surface. Such devices may be referred to in the art as a mouse or a track pad. In some embodiments, the display screen itself can act as a trackpad by sensing the presence and position of one or more fingers on the surface of the display screen. When the cursor is located over a graphical object that appears to be a button or switch, the user can actuate the button or switch by engaging a physical switch on the mouse or trackpad or computer device or tapping the trackpad or touch sensitive display. When the computer detects that the physical switch has been engaged (or that the tapping of the track pad or touch sensitive screen has occurred), it takes the apparent location of the cursor (or in the case of a touch sensitive screen, the detected position of the finger) on the screen and executes the process associated with that location. As an example, not intended to limit the breadth of the disclosed invention, a graphical object that appears to be a two dimensional box with the word “enter” within it may be displayed on the screen. If the computer detects that the switch has been engaged while the cursor location (or finger location for a touch sensitive screen) was within the boundaries of a graphical object, for example, the displayed box, the computer will execute the process associated with the “enter” command. In this way, graphical objects on the screen create a user interface that permits the user to control the processes operating on the computer.

In some instances, especially where the user computer is a mobile computing device used to access data through the network the network may be any type of cellular, IP-based or converged telecommunications network, including but not limited to Global System for Mobile Communications (GSM), Time Division Multiple Access (TDMA), Code Division Multiple Access (CDMA), Orthogonal Frequency Division Multiple Access (OFDM), General Packet Radio Service (GPRS), Enhanced Data GSM Environment (EDGE), Advanced Mobile Phone System (AMPS), Worldwide Interoperability for Microwave Access (WiMAX), Universal Mobile Telecommunications System (UMTS), Evolution-Data Optimized (EVDO), Long Term Evolution (LTE), Ultra Mobile Broadband (UMB), Voice over Internet Protocol (VoIP), Unlicensed Mobile Access (UMA), any form of 802.11.xx or Bluetooth.

Computer program logic implementing all or part of the functionality previously described herein may be embodied in various forms, including, but in no way limited to, a source code form, a computer executable form, scripts and various intermediate forms (e.g., forms generated by an assembler, compiler, linker, or locator.) Source code may include a series of computer program instructions implemented in any of various programming languages (e.g., an object code, an assembly language, or a high-level language such as Javascript, C, C++, JAVA, or HTML or scripting languages that are executed by Internet web-browsers) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The invention may be described in the general context of computer-executable instructions, such as program modules, being executed by a computer. Generally, program modules include routines, programs, objects, binary components that, when executed by the CPU, perform particular tasks or implement particular abstract data types and when running, may generate in computer memory or store on disk, various data structures. A data structure may be represented in the disclosure as a manner of organizing data, but is implemented by storing data values in computer memory in an organized way. Data structures may be comprised of nodes, each of which may be comprised of one or more elements, encoded into computer memory locations into which is stored one or more corresponding data values that are related to an item being represented by the node in the data structure. The collection of nodes may be organized in various ways, including by having one node in the data structure being comprised of a memory location wherein is stored the memory address value or other reference, or pointer, to another node in the same data structure. By means of the pointers, the relationship by and among the nodes in the data structure may be organized in a variety of topologies or forms, including, without limitation, lists, linked lists, trees and more generally, graphs. The relationship between nodes may be denoted in the specification by a line or arrow from a designated item or node to another designated item or node. A data structure may be stored on a mass storage device in the form of data records comprising a database, or as a flat, parsable file. The processes may load the flat file, parse it, and as a result of parsing the file, construct the respective data structure in memory. In other embodiment, the data structure is one or more relational tables stored on the mass storage device and organized as a relational database.

The computer program and data may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed hard disk), an optical memory device (e.g., a CD-ROM or DVD), a PC card (e.g., PCMCIA card, SD Card), or other memory device, for example a USB key. The computer program and data may be fixed in any form in a signal that is transmittable to a computer using any of various communication technologies, including, but in no way limited to, analog technologies, digital technologies, optical technologies, wireless technologies, networking technologies, and internetworking technologies. The computer program and data may be distributed in any form as a removable storage medium with accompanying printed or electronic documentation (e.g., a disk in the form of shrink wrapped software product or a magnetic tape), preloaded with a computer system (e.g., on system ROM or fixed disk), or distributed from a server, website or electronic bulletin board or other communication system (e.g., the Internet or World Wide Web.) It is appreciated that any of the software components of the present invention may, if desired, be implemented in ROM (read-only memory) form. The software components may, generally, be implemented in hardware, if desired, using conventional techniques.

It should be noted that the flow diagrams are used herein to demonstrate various aspects of the invention, and should not be construed to limit the present invention to any particular logic flow or logic implementation. The described logic may be partitioned into different logic blocks (e.g., programs, modules, functions, or subroutines) without changing the overall results or otherwise departing from the true scope of the invention. Oftentimes, logic elements may be added, modified, omitted, performed in a different order, or implemented using different logic constructs (e.g., logic gates, looping primitives, conditional logic, and other logic constructs) without changing the overall results or otherwise departing from the true scope of the invention. Where the disclosure refers to matching or comparisons of numbers, values, or their calculation, these may be implemented by program logic by storing the data values in computer memory and the program logic fetching the stored data values in order to process them in the CPU in accordance with the specified logical process so as to execute the matching, comparison or calculation and storing the result back into computer memory or otherwise branching into another part of the program logic in dependence on such logical process result. The locations of the stored data or values may be organized in the form of a data structure.

The described embodiments of the invention are intended to be exemplary and numerous variations and modifications will be apparent to those skilled in the art. All such variations and modifications are intended to be within the scope of the present invention as defined in the appended claims. Although the present invention has been described and illustrated in detail, it is to be clearly understood that the same is by way of illustration and example only, and is not to be taken by way of limitation. It is appreciated that various features of the invention which are, for clarity, described in the context of separate embodiments may also be provided in combination in a single embodiment. Conversely, various features of the invention which are, for brevity, described in the context of a single embodiment may also be provided separately or in any suitable combination. It is appreciated that the particular embodiment described in the Appendices is intended only to provide an extremely detailed disclosure of the present invention and is not intended to be limiting.

The foregoing description discloses only exemplary embodiments of the invention. Modifications of the above disclosed apparatus and methods which fall within the scope of the invention will be readily apparent to those of ordinary skill in the art. Accordingly, while the present invention has been disclosed in connection with exemplary embodiments thereof, it should be understood that other embodiments may fall within the spirit and scope of the invention as defined by the following claims.

What is claimed:

1. A method executed by a computer system for extracting from a critical communication document data a data value representing a logic state that it was acted upon, said method comprising:
retrieving into the computer system at least one document data comprised of an at least one corresponding text data comprising an at least one medical data;
using a first word statistical frequency analysis process to automatically extract a corresponding at least one first set of at least one keywords from the at least one text data;
using the extracted at least one first set of at least one keywords to automatically determine whether the at least one document data is comprised of a corresponding at least one critical communication document;
using a second word statistical frequency analysis process to automatically extract from the at least one critical communication document data a corresponding at least one data value representing the logic state that the at least one critical communication was acted upon; and
storing the extracted data value in computer memory.

2. The method of claim 1 where the first or second word statistical frequency analysis process is comprised of using the text data as input into a word frequency detection process that automatically determines a most frequently used word in the text data that is a member of a predetermined set of keywords.

3. The method of claim 1 where the first or second word statistical frequency analysis process is comprised of:
automatically converting the text data into a set of words comprising the text data;
automatically determining a corresponding set of statistical frequencies of the converted words in the text data; and
automatically selecting a subset of the set of converted words in dependence on a set of frequencies corresponding to the words comprising the text data.

4. The method of claim 1 where the first or second word statistical frequency analysis process is comprised of:
determining an at least one corresponding statistical frequency for an at least one word comprising the text data; and
using a best-fit analysis to determine which of a predetermined group of keywords exhibit a corresponding predetermined frequency of use that sufficiently matches the determined corresponding at least one statistical frequency of the at least one words comprising the text data.

5. The method of claim 4 where the step of using a best fit analysis is comprised of using an R square calculation applied to the word frequencies in the text data.

6. The method of claim 4 where the step of using a best fit analysis is comprised of using a linear regression of the word frequencies in the text data and comparing the linear regression result to a linear regression applied to a predetermined set of words and corresponding word frequencies.

7. The method of claim 4 where the step of using a best fit analysis is comprised of calculating a correlation between of the word frequencies in the text data and to a predetermined set of words and corresponding word frequencies.

8. The method of claim 2 where the first or second word statistical frequency analysis process is comprised of:
parsing the at least one document data to detect at least one word comprising the text data where the at least one word is present in repetitive patterns with the text data.

9. The method of claim 1 where the first or second word statistical frequency analysis process is comprised of using a natural language text analysis process that uses the at least one document data as input.

10. The method of claim 1 where the first word statistical frequency analysis process is comprised of extracting at least one subject matter keyword from the text data by using a statistical analysis of at least one words comprising the text data to identify a statistical pattern of word usage that sufficiently matches one of at least one pre-determined statistical patterns of word usage that corresponds to a pre-determined subject matter keyword.

11. The method of claim 1 further comprising:
parsing the at least one document data to extract a sender identifier data, a receiver identifier data and a subject matter identifier data.

12. The method of claim 11 further comprising:
parsing the at least one document data to extract a time that the critical communication message comprising the critical communication document was transmitted, a time when the message when was received; and
generating a database record comprised of database entries representing the time that the message was sent, the time when the message when was received, the sender identity, the recipient identity and the subject matter identifier.

13. The method of claim 11 further comprising:
automatically generating a report data comprised of a first at least one identifier data corresponding to the at least one referring clinician identifiers, a second at least one identifier data corresponding to the at least one corresponding reporting clinician, and a corresponding acted upon data value.

14. The method of claim 1 further comprising:
using a natural language text analysis process to determine the value representing that the at least one critical communication was acted upon.

15. The method of claim 1 further comprising: automatically processing the at least one text data to make a determination whether the corresponding critical communication document is comprised of data indicating that a reporting clinician communicated a critical medical finding to a referring clinician.

16. The method of claim 14 where the using a natural language text analysis process is comprised of:
determining the at least one key word in the at least one document by natural language parsing of text data comprising the at least one document.

17. A system comprised of a computer system for extracting from a critical communication document data a data value representing a logic state that it was acted upon, said computer system comprised of program data stored in computer memory that when executed causes the system to:
retrieve into the computer system at least one document data comprised of an at least one corresponding text data comprising an at least one medical data;
use a first word statistical frequency analysis process to automatically extract a corresponding at least one first set of at least one keywords from the at least one text data;
use the extracted at least one first set of at least one keywords to automatically determine whether the at least one document data is comprised of a corresponding at least one critical communication document;
use a second word statistical frequency analysis process to automatically extract from the at least one critical communication document data a corresponding at least one data value representing the logic state that the at least one critical communication was acted upon; and
store the extracted data value in computer memory.

18. The system of claim 17 where the first or second word statistical frequency analysis process is comprised of using the text data as input into a word frequency detection process that automatically determines a most frequently used word in the text data that is a member of a predetermined set of keywords.

19. The system of claim 17 where the first or second word statistical frequency analysis process is comprised of:
automatically converting the text data into a set of words comprising the text data;
automatically determining a corresponding set of statistical frequencies of the converted words in the text data; and
automatically selecting a subset of the set of converted words in dependence on a set of frequencies corresponding to the words comprising the text data.

20. The system of claim 17 where the first or second word statistical frequency analysis process is comprised of:
determining an at least one corresponding statistical frequency for an at least one word comprising the text data; and
using a best-fit analysis to determine which of a predetermined group of keywords exhibit a corresponding predetermined frequency of use that sufficiently matches the determined corresponding at least one statistical frequency of the at least one words comprising the text data.

21. The system of claim 20 where the step of using a best fit analysis is comprised of using an R square calculation applied to the word frequencies in the text data.

22. The system of claim 20 where the step of using a best fit analysis is comprised of using a linear regression of the word frequencies in the text data and comparing the linear regression result to a linear regression applied to a predetermined set of words and corresponding word frequencies.

23. The system of claim 20 where the step of using a best fit analysis is comprised of calculating a correlation between of the word frequencies in the text data and to a predetermined set of words and corresponding word frequencies.

24. The system of claim 20 where the first or second word statistical frequency analysis process is comprised of:
parsing the at least one document data to detect at least one word comprising the text data where the at least one word is present in repetitive patterns with the text data.

25. The system of claim 20 where the first or second word statistical frequency analysis process is comprised of using a natural language text analysis process that uses the at least one document data as input.

26. The system of claim 20 where the first word statistical frequency analysis process is comprised of extracting at least one subject matter keyword from the text data by using a statistical analysis of at least one words comprising the text data to identify a statistical pattern of word usage that sufficiently matches one of at least one pre-determined statistical patterns of word usage that corresponds to a pre-determined subject matter keyword.

27. The system of claim 26 where the program data further causes the system to:
parse the at least one document data to extract a sender identifier data, a receiver identifier data and a subject matter identifier data.

28. The system of claim 27 where the program data further causes the system to:
parse the at least one document data to extract a time that the critical communication message comprising the critical communication document was transmitted, a time when the message when was received; and
generate a database record comprised of database entries representing the time that the message was sent, the time when the message when was received, the sender identity, the recipient identity and the subject matter identifier.

29. The system of claim 27 where the program data further causes the system to:
automatically generate a report data comprised of a first at least one identifier data corresponding to the at least one referring clinician identifiers, a second at least one identifier data corresponding to the at least one corresponding reporting clinician, and a corresponding acted upon data value.

30. The system of claim 23 where the program data further causes the system to:
use a natural language text analysis process to determine the value representing that the at least one critical communication was acted upon.

31. The system of claim 23 where the program data further causes the system to:
automatically processing the at least one text data to make a determination whether the corresponding critical communication document is comprised of data indicating that a reporting clinician communicated a critical medical finding to a referring clinician.

32. The system of claim 31 where the using a natural language text analysis process is comprised of:
determining the at least one key word in the at least one document by natural language parsing of text data comprising the at least one document.
